(19) United States
(12) Patent Application Publication
(10) Pub. No.: US 2024/0207338 A1
(43) Pub. Date: Jun. 27, 2024

(54) KETAMINE AND CANNABIS FOR THE TREATMENT OF EMOTIONAL DISORDERS

(71) Applicants: Paul Freeman DALEY, El Sobrante, CA (US); Progressive Therapeutics Inc, Orleans, MA (US)

(72) Inventors: Philip E. WOLFSON, San Anselmo, CA (US); Paul Freeman DALEY, El Sobrante, CA (US)

(21) Appl. No.: 18/288,870

(22) PCT Filed: May 2, 2022
(86) PCT No.: PCT/US2022/027313

§ 371 Date: Oct. 30, 2023

Related U.S. Application Data
(60) Provisional application No. 63/182,261, filed on Apr. 30, 2021.

(51) Int. Cl. A61K 36/185; A61K 9/00; A61K 31/00; A61K 31/135; A61P 25/22; A61P 25/24

(52) U.S. Cl. CPC A61K 36/185; A61K 9/0056; A61K 9/007; A61K 31/135; A61K 31/658; A61P 25/22; A61P 25/24

(57) ABSTRACT

The present invention features methods and compositions for the treatment of an emotional disorder in a subject by administering ketamine, norketamine, 6-hydroxynorketamine, or a pharmaceutically acceptable salt thereof, and cannabis or a cannabinoid in an amount that is sufficient to treat the emotional disorder.

### BACKGROUND OF THE INVENTION

The invention relates to the field of methods for the treatment of emotional disorders and other medical entities with symptoms that are related to emotional disorders or are of a somatic nature. Nonlimiting examples of emotional disorders include anxiety, depression, somatization, and other categories of emotional disorders as per the DSM-5-R. Anxiety disorders are the most commonly diagnosed mental illness in the United States. Nonlimiting examples of anxiety disorders include Generalized Anxiety Disorder (GAD), Panic Disorder (PD), Social Anxiety Disorder (SAD), Obsessive-Compulsive Disorder (OCD), Specific Phobic Disorders (SPD), Post Traumatic Stress Disorder (PTSD), Agoraphobia, or Separation Anxiety Disorder (SeAD). Nonlimiting examples of depressive disorders include Dysthymia, Major Depression, Bipolar II Disorder, Postpartum Depression, Adjustment with Depression, and other depressive disorders. Nonlimiting examples of physical symptoms that may be treated by the above method include headaches; migraines; physical symptoms related to the menstrual cycle such as breast tenderness, cramps, headaches; symptoms of somatization disorders; fibromyalgia, various neurologic symptoms and disorders, and pain from a wide variety of causes.

A significant portion of emotional disorders, including anxiety disorders and depressive disorders, are refractory to currently employed methods of treatment.

Applicants have discovered that the concurrent administration of ketamine and cannabis can be useful for treating emotional disorders. Ketamine's many decades long use has established its safety. In the methods described herein, dosages are much lower than that used for anesthesia and side effects are minimal. Cannabis is a medicinal plant that has been consumed by humans for millenia. The therapeutic benefits of cannabis are currently recognized by a majority of U.S. states and the District of Columbia, which have legalized the medicinal use of cannabis.

### SUMMARY OF THE INVENTION

The invention features a method of treating an emotional disorder in a subject in need thereof, the method including administering to the subject (i) ketamine, norketamine, 6-hydroxynorketamine, or a pharmaceutically acceptable salt thereof, and (ii) cannabis or a cannabinoid each in an amount that together is sufficient to treat the emotional disorder.

In a first embodiment of the above method, the emotional disorder is an anxiety disorder. The anxiety disorder can be selected from, or generalized to include, Generalized Anxiety Disorder (GAD), Panic Disorder (PD), Social Anxiety Disorder (SAD), Obsessive-Compulsive Disorder (OCD), Specific Phobic Disorders (SPD), Post Traumatic Stress Disorder (PTSD), Agoraphobia, or Separation Anxiety Disorder (SeAD).

In a second embodiment of the above method, the emotional disorder is a depressive disorder. The depressive disorder can be selected from, or generalized to include, Dysthymia, Major Depression, Bipolar II Disorder, Postpartum Depression, or Adjustment Disorder with Depression.

In a specific of the above method, the invention provides a method for treating a variety of physical symptoms that may or may not be related to an emotional disorder. Nonlimiting examples of physical symptoms that may be treated by the above method include headaches; migraines; physical symptoms related to the menstrual cycle such as breast tenderness, cramps, headaches; symptoms of somatization disorders; fibromyalgia, various neurologic symptoms and disorders, and pain from a wide variety of causes.

In some embodiments of the above method, the cannabis is cannabis plant matter, e.g., the bud or leaves of a plant of a Cannabis indica species, Cannabis sativa species, Cannabis ruderalis species, or hybrid cannabis subspecies. In some embodiments, the cannabis plant matter is smoked by a subject, e.g., in a cannabis containing cigarette or from a pipe or other device. In some embodiments, the cannabis plant matter is be vaporized by a subject. In some embodiments, the cannabis is an extract of cannabis plant material that contains at least one cannabinoid compound, e.g., a concentrate or refined oil. In some embodiments, the oil and/or extract is vaporized by a subject. In some embodiments, the oil and/or concentrate is smoked by a subject. In some embodiments, the cannabis is formulated in a topical composition. In some embodiments, the cannabis is formulated into a tablet or capsule. In some embodiments, the cannabis is included in a foodstuff or a beverage. In some embodiments, the cannabis is produced from a cannabis sub-species that is high in tetrahydrocannabinol (THC) content, (e.g., contains at least 20% THC by dry weight; e.g., at least 20% 21%, 22%, 23%, 24%, 25%, or more). In some embodiments, the cannabis is a high CBD Cannabis sub-species (e.g., contains at least 1%, CBD by dry weight, e.g., at least 1% at least 2%, 3%, 4%, 5%, or more etc.). In some embodiments, the THC is (−)-trans-Δ⁹-tetrahydrocannabinol (delta-9-THC) or (−)-trans-Δ⁸-tetrahydrocannabinol (delta-8-THC). In some embodiments the cannabinoid is (−)-trans-Δ⁹-tetrahydrocannabivarin (delta-9-THCV) or (−)-trans-Δ⁸-tetrahydrocannabivarin (delta-8-THCV). In some embodiments the cannabinoid is cannabinol. In some cases the cannabinoid is cannabivarin (CBV).

In one embodiment of any of the above methods, the method can include initiating treatment following the appearance of an emotional disorder in the subject.

In any of the above methods the ketamine, norketamine, 6-hydroxynorketamine, or a pharmaceutically acceptable salt thereof, and cannabis product or a cannabinoid are concurrently administered once or more daily, once or more every other day, or once or more every three days, or depending on the presence and/or severity of the emotional disorder. For example, the concurrent administration of the ketamine, norketamine, 6-hydroxynorketamine, or a pharmaceutically acceptable salt thereof, and cannabis or a cannabinoid can be once or twice daily for a period of from 1 to 30 days, preferably for an interval of 10 days or fewer, with subsequent repetition as required clinically (e.g., for a period of from 1 to 30 days, 2 to 29 days, 3 to 27 days, 4 to 26 days, 5 to 25 days, 6 to 24 days, 7 to 23 days, 8 to 22 days, 9 to 21 days, 10 to 20 days, 11 to 19 days, 12 to 18 days, 13 to 17 days, 14 to 16 days, or for a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, or for a period of 30 days or fewer, 29 days or fewer, 28 days or fewer, 27 days or fewer, 26 days or fewer, 25 days or fewer, 24 days or fewer, 23 days or fewer, 22 days or fewer, 21 days or fewer, 20 days or fewer, 19 days or fewer, 18 days or fewer, 17 days or fewer, 16 days or fewer, 15 days or fewer, 14 days or fewer, 13 days or fewer, 12 days or fewer, 11 days or fewer, 10 days or fewer, 9 days or fewer, 8 days or fewer, 7 days or fewer, 6 days or fewer, 5 days or fewer, 4 days or fewer, 3 days or fewer, or 2 days or fewer), followed by a period of at least one or two weeks during which no ketamine, norketamine, 6-hydroxynorketamine, or a pharmaceutically acceptable salt thereof, and cannabis or a cannabinoid is concurrently administered to the subject. In particular embodiments, the concurrent administration is once or more daily, or intermittently for a period of from 1 to 30 days, followed by a variable period of time during which no ketamine, norketamine, 6-hydroxynorketamine, or a pharmaceutically acceptable salt thereof, and cannabis or a cannabinoid is concurrently administered to the subject. While the embodiment may be administered prior to sleep, it will not be administered during the sleep cycle. It will not be administered daily for more than 4 weeks at a time, though generally less frequently and not continuously on any particular day, unless there is clinical support for such utilization. In particular embodiments, the ketamine, norketamine, 6-hydroxynorketamine, or a pharmaceutically acceptable salt thereof, and cannabis or a cannabinoid is concurrently administered to the subject only in the evening. In particular embodiments, the ketamine, norketamine, 6-hydroxynorketamine, or a pharmaceutically acceptable salt thereof, and cannabis or a cannabinoid is concurrently administered to the subject once or more daily in the evening. In particular embodiments, the ketamine, norketamine, 6-hydroxynorketamine, or a pharmaceutically acceptable salt thereof, and cannabis or a cannabinoid is concurrently administered to the subject during the daytime one or more times. In particular embodiments, the ketamine, norketamine, 6-hydroxynorketamine, or a pharmaceutically acceptable salt thereof, and cannabis or a cannabinoid is concurrently administered to the subject one or more times daily and/or in the evening.

In particular embodiments of any of the above methods, the method includes administering an average daily dose of from 1 mg to 500 mg (e.g., 10 mg to 200 mg, 25 mg to 150 mg, 35 mg to 125 mg, 50 mg to 250 mg, or 100 mg to 500 mg) of ketamine, or a pharmaceutically acceptable salt thereof, to the subject. For example, the method can include administering an average daily dose of from 10 mg to 200 mg or 100 mg to 500 mg (e.g., 30±20 mg, 60±20 mg, 90±20 mg, 150±50 mg, 250±50 mg, 350±50 mg, or 450±50 mg) of enantiomerically pure S-(+)-ketamine, or a pharmaceutically acceptable salt thereof, to the subject. In particular embodiments, the method includes administering an average daily dose of from 10 mg to 200 mg or 100 mg to 500 mg (e.g., 30±20 mg, 60±20 mg, 90±20 mg, 150±50 mg, 250±50 mg, 350±50 mg, or 450±50 mg) of enantiomerically pure R-(−)-ketamine, or a pharmaceutically acceptable salt thereof, to the subject. In some embodiments, the method includes administering an average daily dose of from 10 mg to 200 mg or 100 mg to 500 mg (e.g., 30±20 mg, 60±20 mg, 90±20 mg, 150±50 mg, 250±50 mg, 350±50 mg, or 450±50 mg) of racemic ketamine, or a pharmaceutically acceptable salt thereof, to the subject.

In some embodiments of any of the above methods, the method includes administering one or more doses of from 1 mg to 500 mg (e.g., 10 mg to 200 mg, 25 mg to 150 mg, 35 mg to 125 mg, 50 mg to 250 mg, or 100 mg to 500 mg) of norketamine, or a pharmaceutically acceptable salt thereof, to the subject. For example, the method can include administering an average daily dose of from 10 mg to 200 mg (e.g., 30±20 mg, 60±20 mg, 90±20 mg, or 150±50 mg) of enantiomerically pure S-(+)-norketamine, or a pharmaceutically acceptable salt thereof, to the subject. In particular embodiments, the method includes administering an average daily dose of from 10 mg to 200 mg (e.g., 30±20 mg, 60±20 mg, 90±20 mg, or 150±50 mg) of enantiomerically pure R-(−)-norketamine, or a pharmaceutically acceptable salt thereof, to the subject. In some embodiments, the method includes administering an average daily dose of from 10 mg to 200 mg (e.g., 30±20 mg, 60±20 mg, 90±20 mg, or 150±50 mg) of racemic norketamine, or a pharmaceutically acceptable salt thereof, to the subject.

In some embodiments of any of the above methods, the method includes administering one or more doses of from 1 mg to 500 mg (e.g., 10 mg to 200 mg, 25 mg to 150 mg, 35 mg to 125 mg, 50 mg to 250 mg, or 100 mg to 500 mg) of 6-hydroxynorketamine, or a pharmaceutically acceptable salt thereof, to the subject. For example, the method can include administering an average daily dose of from 10 mg to 200 mg (e.g., 30±20 mg, 60±20 mg, 90±20 mg, or 150±50 mg) of isomerically pure (2R,6R)-6-hydroxynorketamine, or a pharmaceutically acceptable salt thereof, to the subject. In particular embodiments, the method includes administering an average daily dose of from 10 mg to 200 mg (e.g., 30±20 mg, 60±20 mg, 90±20 mg, or 150±50 mg) isomerically pure (2S,6S)-6-hydroxynorketamine, or a pharmaceutically acceptable salt thereof, to the subject.

In particular embodiments of any of the above methods, the administration of ketamine, norketamine, 6-hydroxynorketamine, or a pharmaceutically acceptable salt thereof is by a route selected from oral, sublingual, intranasal, intramuscular, intravenous, transdermal, vaginal, and rectal administration, or any administration route described herein.

In a particular embodiment of the above methods, the ketamine, norketamine, 6-hydroxynorketamine, or a pharmaceutically acceptable salt thereof is formulated in a lozenge.

In particular embodiments of any of the above methods, the administration of cannabis or a cannabinoid is by a route selected from oral, pulmonary, or transdermal administration, or any administration route described herein.

In particular embodiments of any of the above methods, the ketamine, norketamine, 6-hydroxynorketamine, or a pharmaceutically acceptable salt thereof, is administered in an amount, or in a dosage form (e.g., a sustained release dosage form), that, upon administration to the subject, does not result in anesthesia in the subject.

In particular embodiments of the above methods, embodiments, administration of ketamine norketamine, 6-hydroxynorketamine, or a pharmaceutically acceptable salt thereof with cannabis or a cannabinoid ameliorates the anesthetic effect of ketamine norketamine, 6-hydroxynorketamine, or a pharmaceutically acceptable salt thereof relative to the anesthetic effect caused by administration of ketamine norketamine, 6-hydroxynorketamine, or a pharmaceutically acceptable salt thereof alone. For example, subjects administered ketamine, norketamine, 6-hydroxynorketamine, or a pharmaceutically acceptable salt thereof alone may experience reduced sedation observable as a score lower on a scale intended to measure sedation, e.g., the Richmond Agitation-Sedation Scale, relative to subjects administered ketamine, norketamine, 6-hydroxynorketamine, or a pharmaceutically acceptable salt thereof in combination with cannabis.

In certain embodiments of the methods of the invention the ketamine, norketamine, 6-hydroxynorketamine, or a pharmaceutically acceptable salt thereof, and the cannabis or a cannabinoid are administered separately within 60 minutes, 30 minutes, or 15 minutes of each other.

In some embodiments, the ketamine, norketamine, 6-hydroxynorketamine, or a pharmaceutically acceptable salt thereof, and the cannabis or a cannabinoid are administered concurrently. In particular embodiments, the ketamine, norketamine, 6-hydroxynorketamine, or a pharmaceutically acceptable salt thereof, and the cannabis or a cannabinoid are formulated together and administered simultaneously. In particular embodiments, the method includes (i) the ketamine, norketamine, 6-hydroxynorketamine, or a pharmaceutically acceptable salt thereof, and the cannabis or a cannabinoid are formulated together in a sublingual dosage form; and (ii) the sublingual dosage form is administered sublingually. In certain embodiments, the sublingual dosage form is selected from films, strips, lozenges, and orally dissolving tablets.

In some embodiments, the cannabis or cannabinoid comprises at least one of delta-9-THC, delta-8-THC, cannabinol (CBN), delta-9-THCV, delta-8-THCV, cannabidiol (CBD), and cannabivarin (CBV).

In some embodiments of any of the above methods, the method includes administering one or more doses of from 1 mg to 40 mg (e.g., 1 mg to 5 mg, 2 mg to 15 mg, 5 mg to 20 mg, 7 mg to 25 mg, or 10 mg to 40 mg) of delta-9-THC to the subject. In particular embodiments, the method includes administering an average daily dose of from 2 mg to 100 mg (e.g., 4±2 mg, 7.5±2.5 mg, 15±5 mg, 25±30 5 mg, 35±5 mg, 45±5 mg, 55±5 mg, 65±5 mg, or 75±25 mg) delta-9-THC to the subject.

In some embodiments of any of the above methods, the method includes administering one or more doses of from 1 mg to 80 mg (e.g., 1 mg to 5 mg, 2 mg to 15 mg, 5 mg to 20 mg, 7 mg to 25 mg, 10 mg to 40 mg, 20 mg to 50 mg, 40 mg to 70 mg, 50 mg to 80 mg) of delta-8-THC to the subject. In particular embodiments, the method includes administering an average daily dose of from 2 mg to 100 mg (e.g., 4±2 mg, 7.5±2.5 mg, 15±5 mg, 25±5 mg, 35±5 mg, 45±5 mg, 55±5 mg, 65±5 mg, or 75±25 mg) delta-8-THC to the subject.

In some embodiments of any of the above methods, the method includes administering one or more doses of from 1 mg to 30 mg (e.g., 1 mg to 5 mg, 2 mg to 15 mg, 5 mg to 20 mg, 7 mg to 25 mg, or 10 mg to 30 mg) of CBN to the subject. In particular embodiments, the method includes administering an average daily dose of from 2 mg to 100 mg (e.g., 4±2 mg, 7.5±2.5 mg, 15±5 mg, 25±5 mg, 35±5 mg, 45±5 mg, 55±5 mg, 65±5 mg, or 75±25 mg) CBN to the subject.

In some embodiments of any of the above methods, the method includes administering one or more doses of from 1 mg to 30 mg (e.g., 1 mg to 5 mg, 2 mg to 15 mg, 5 mg to 20 mg, 7 mg to 25 mg, or 10 mg to 30 mg) of delta-9-THCV to the subject. In particular embodiments, the method includes administering an average daily dose of from 2 mg to 100 mg (e.g., 4±2 mg, 7.5±2.5 mg, 15±5 mg, 25±30 5 mg, 35±5 mg, 45±5 mg, 55±5 mg, 65±5 mg, or 75±25 mg) delta-9-THCV to the subject.

In some embodiments of any of the above methods, the method includes administering one or more doses of from 1 mg to 30 mg (e.g., 1 mg to 5 mg, 2 mg to 15 mg, 5 mg to 20 mg, 7 mg to 25 mg, or 10 mg to 30 mg) of delta-8-THCV to the subject. In particular embodiments, the method includes administering an average daily dose of from 2 mg to 100 mg (e.g., 4±2 mg, 7.5±2.5 mg, 15±5 mg, 25±5 mg, 35±5 mg, 45±5 mg, 55±5 mg, 65±5 mg, or 75±25 mg) delta-8-THCV to the subject.

In some embodiments of any of the above methods, the method includes administering one or more doses of from 10 mg to 800 mg (e.g., 10 mg to 50 mg, 20 mg to 150 mg, 50 mg to 200 mg, 70 mg to 250 mg, 100 mg to 400 mg, 200 mg to 500 mg, 400 mg to 700 mg, 500 mg to 800 mg) of CBD to the subject. In particular embodiments, the method includes administering an average daily dose of from 20 mg to 1,000 mg (e.g., 40±20 mg, 75±25 mg, 150±50 mg, 250±50 mg, 350±50 mg, 450±50 mg, 550±50 mg, 650±50 mg, or 750±250 mg) CBD to the subject.

In some embodiments of any of the above methods, the method includes administering one or more doses of from 1 mg to 30 mg (e.g., 1 mg to 5 mg, 2 mg to 15 mg, 5 mg to 20 mg, 7 mg to 25 mg, or 10 mg to 30 mg) of CBV to the subject. In particular embodiments, the method includes administering an average daily dose of from 2 mg to 100 mg (e.g., 4±2 mg, 7.5±2.5 mg, 15±5 mg, 25±5 mg, 35±5 mg, 45±5 mg, 55±5 mg, 65±5 mg, or 75±25 mg) CBV to the subject.

### Definitions

As used herein, the term “average daily dose” refers to the average amount of ketamine, norketamine, 6-hydroxynorketamine, or a pharmaceutically acceptable salt thereof, and cannabis or a cannabinoid concurrently administered to a subject for a given dosing regimen. For example, single doses of 100 mg of ketamine administered every other day is an average daily dose of 50 mg. For a regimen in which 15 mg doses of ketamine are administered twice daily, the average daily dose is 30 mg. As used herein, “beverage” refers to a fluid suitable for consumption, such as a tea, soda, or soft drink.

As used herein, “cannabis” refers to the material of any species or subspecies of cannabis plant, in particular the flower buds of a cannabis plant of any species or subspecies, as well as any product or substance manufactured from the material of a cannabis plant that contains at least one cannabinoid compound.

As used herein, “cannabis oil” refers to a liquid mixture of compounds obtained from the extraction of cannabis plants. Such compounds include, but are not limited to, cannabinoids. The exact composition of cannabis oil will depend on the strain of cannabis used for extraction, the efficiency and process of the extraction itself, and any additives that might be incorporated to alter the palatability or improve administration of the cannabis oil.

As used herein, “cannabis concentrate” refers to a solid, semi-solid, or waxy mixture of compounds obtained from the extraction of cannabis plants. Such compounds include, but are not limited to, cannabinoids. The exact composition of cannabis concentrate will depend on the strain of cannabis used for extraction and the efficiency and process of the extraction itself.

As used herein, “cannabinoid” refers to a class of chemical compounds that act on the cannabinoid receptors. Cannabinoids found in cannabis include, without limitation: canaigre, cannabichromene (CBC), cannabidiol (CBD), tetrahydrocannabinol (THC) (e.g., delta-9-THC or delta-8-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA).

As used herein, “foodstuff” refers to an edible substance such as a baked good (e.g., a cookie or brownie), a candy, a snack, or a meal (e.g., breakfast, lunch, or dinner).

As used herein, “high-THC subspecies” refers to a cannabis subspecies strain having 20% or more THC by dry weight.

As used herein, “high-CBD subspecies” refers to a cannabis subspecies having 1% or more CBD by dry weight.

As used herein, the term “pharmaceutically acceptable salts” refers to salts of the active compounds of the invention that can be prepared in situ during the final isolation and purification of the compounds of the invention, or separately by reacting the free base function with a suitable organic or inorganic acid, or separately by reacting an acid function with a suitable organic or inorganic base.

A “therapeutically effective amount” or “an amount sufficient” of a drug is an amount effective to demonstrate a desired activity of the drug. According to the instant invention, a therapeutically effective amount of ketamine and/or cannabis or a cannabinoid is an amount effective to alleviate, i.e., noticeably reduce, the symptoms of an emotional disorder. The therapeutically effective of ketamine or cannabis or a cannabinoid, when administered in combination, may be less than the therapeutically effective amount of either ketamine or cannabis or a cannabinoid when administered alone.

As used herein, a “topical composition” means, for example, an ointment, cream, lotion, paste, gel, etc. which releases at least one compound at a predetermined rate over a defined period of time to a defined site of application.

As used herein, the term “treating” refers to administering a pharmaceutical composition for therapeutic purposes. To “treat disease” or use for “therapeutic treatment” refers to administering treatment to a subject already suffering from a condition to improve or stabilize the subject's condition.

As used herein, the term “vaporized” means converted into a vapor and/or aerosol by the application of heat.

### DETAILED DESCRIPTION

The invention for the first time provides a method of treating an emotional disorder by the concurrent administration of ketamine and cannabis or a cannabinoid. The invention further provides a method for treating the physical symptoms of an emotional disorder by the concurrent administration of ketamine and cannabis or a cannabinoid. The emotional disorder treated by the method may be an anxiety disorder. The emotional disorder treated by the method may be a depressive disorder.

The compositions of the invention may be designed to be short-acting, fast-releasing, long-acting, or sustained-releasing as described herein. Thus, the pharmaceutical formulations may also be formulated for controlled release or for slow release.

Anxiety is broadly defined as a state of unwarranted or inappropriate worry often accompanied by restlessness, tension, distraction, irritability and sleep disturbances. This disproportionate response to environmental stimuli can hyperactivate the hypothalamic-pituitary-adrenal axis and the autonomic nervous system, resulting in somatic manifestation of anxiety, including shortness of breath, sweating, nausea, rapid heartbeat and elevated blood pressure. Anxiety disorders represent a range of conditions and as a result have been classified into multiple distinct conditions, including Generalized Anxiety Disorder (GAD), Panic Disorder (PD), Social Anxiety Disorder (SAD), Obsessive-Compulsive Disorder (OCD), Specific Phobic Disorders (SPD), Post Traumatic Stress Disorder (PTSD), Agoraphobia, or Separation Anxiety Disorder (SeAD). GAD is the most commonly occurring of the anxiety disorders and is characterized by excessive and persistent worries. In the general population the lifetime prevalence rate of GAD ranges from 4.1 to 6.6% with somewhat higher rates in women than in men. The individual with GAD worries about life events such as marital relationships, job performance, health, money, and social status. Individuals with GAD startle easily and may suffer from depression. Some of the specific symptoms of GAD include restlessness, motor tension, difficulty concentrating, and irritability. The severity of the symptoms over time may be linked to the changing nature of the environmental stressor. With increasing age, GAD symptoms become less severe. PD is a well-studied psychiatric condition that consists of multiple disabling panic attacks characterized by an intense autonomic arousal. In addition, heightened fear and anxiety states occur both during and between panic attacks. Approximately 3% of women and 1.5% of men have panic attacks. During a panic attack, the individual experiences multiple symptoms including light-headedness, a pounding heart and difficulty in breathing. SAD is a chronic disorder in which social interactions cause irrational anxiety. Individuals suffering from SAD may experience heightened anxiety, fear, self-consciousness, and embarrassment during everyday social interactions. Individuals suffering from SAD may experience fear of being humiliated, judged, and/or rejected during everyday social interactions. OCD is a common, chronic, and long-lasting disorder characterized by unreasonable, recurring thoughts and fears (e.g., obsessions) that lead to compulsive behaviors. Individuals suffering from OCD may experience unreasonable fear of germs or contamination, unwanted forbidden or taboo thoughts, aggressive thoughts towards themselves or others, or an obsessive desire to have things in a symmetrical or perfect order. Individuals suffering from OCD may engage in compulsive behaviors. Exemplary, nonlimiting compulsive behaviors include excessive cleaning and/or handwashing, ordering or arranging things in a particular, precise fashion, repeatedly checking whether a task is completed (e.g., whether a door is locked or an oven is off) or compulsive counting. SPD is a class of disorders characterized by persistent, unrealistic, intense anxiety about and fear of specific situations, circumstances, and objects. Nonlimiting examples of situations, circumstances, and objects that individuals suffering from SPD may have anxiety about or fear of include animals, heights, or thunderstorms. PTSD is an anxiety disorder that occurs in people who have experienced or witnessed a traumatic event. Nonlimiting examples of traumatic events that may cause PTSD in an individual include a serious accident, a terrorist act, war and/or combat, sexual violence, or the imminent threat of death, sexual violence, or serious injury. Individuals suffering from PTSD may experience intense, disturbing thoughts or feelings related to their traumatic experience which last long after the traumatic event has ended. Nonlimiting examples of intense, disturbing thoughts or feelings include flashbacks or nightmares, feelings of sadness, fear, or anger, and feelings of detachment and/or estrangement from other people. Agoraphobia is an anxiety disorder characterized by the fear of situations which are perceived to be unsafe and inescapable. Nonlimiting examples of situations individual suffering from agoraphobia may be afraid of include traveling on public transportation, visiting a shopping center, being in a crowd, being in an open space, being in an enclosed space, standing in line, or leaving their home. SeAD is an anxiety disorder characterized by excessive fear, anxiety, and distress about being away from home or loved ones. Nonlimiting examples of fears an individual suffering from SeAD may experience include fear of losing a parent or other loved one to an illness or disaster, or fear that something bad will happen in the future that will lead to separation from home and/or loved ones (e.g., becoming lost or being kidnapped).

Depressive disorders are broadly defined as serious mood disorders characterized by sadness severe enough or persistent enough to interfere with function and often by decrease interest in pleasure in activities. The origin of depressive disorders likely involves a combination of heredity factors, changes in neurotransmitter levels, altered neuroendocrine function, and psychosocial factors. Depressive disorders represent a range of conditions and as a result have been classified into multiple distinct conditions, including but not limited to Dysthymia, Major Depression, Bipolar II Disorder, and Adjustment Disorder with Depression. Dysthymia, also referred to as Persistent depressive disorder (PDD), is a chronic depressive disorder characterized by symptoms of depression that last for at least 2 years. Nonlimiting examples of symptoms an individual suffering from Dysthymia may experience include poor appetite or overeating, changes in sleep patterns (e.g., insomnia or hypersomnia), low energy or fatigue, low self-esteem, poor concentration or difficulty making decisions, or feelings of hopelessness. Major Depression is depressive mood disorder characterized by persistently depressed mood causing a significant impairment in daily life. Nonlimiting examples of symptoms an individual suffering from Major Depression may experience include depressed mood, loss of interest or pleasure in almost all activities, significant, unintentional weight gain or loss (e.g., >5% change in body weight in one month), sleep disturbance (e.g., insomnia or hypersomnia), psychomotor changes (e.g., agitation or retardation) severe enough to be observable by others, tiredness, fatigue, or low energy, decreased efficiency with which routine tasks are completed, a sense of worthlessness or inappropriate, delusional guilt, impaired ability to think, concentrate, or make decisions, recurrent thoughts of death, suicidal ideation, or suicide attempts. Bipolar II Disorder is a depressive disorder characterized by a pattern of depressive episodes and hypomanic episodes. Nonlimiting examples of symptoms an individual suffering from Bipolar II Disorder may experience during a depressive episode include feelings of sadness, emptiness, or hopelessness, low motivation, loss of interest in activities, sleep disturbance (e.g., insomnia or hypersomnia), low energy, feelings of worthlessness or guilt, difficulty focusing, unintentional weight loss or gain, or suicidal thoughts. Nonlimiting examples of symptoms an individual suffering from Bipolar II Disorder may experience during a hypomanic episode include increased energy, activity, or agitation, an exaggerated sense of well-being and self-confidence, decreased need for sleep, unusual talkativeness, racing thoughts, distractibility or poor decision making. Adjustment Disorder with Depression is a depressive disorder characterized by the development of emotional or behavioral symptoms in response to an identifiable stressor occurring within 3 months of the onset of the stressor. Nonlimiting examples of symptoms an individual suffering from Adjustment Disorder with Depression may experience include low mood, tearfulness, or feelings of hopelessness.

Individuals suffering from an emotional disorder, e.g., an anxiety disorder and/or a depressive disorder, may experience physical symptoms of the disorder. Nonlimiting examples of physical symptoms experienced by individuals suffering from an emotional disorder include body pains, headaches, joint pains, nausea, vomiting, fatigue, weakness, numbness, shortness of breathing, or shortness of breath.

Ketamine is an inexpensive, readily available drug, with minor adverse side effects. Thus, the invention contemplates additional savings to the overburdened health care system. Sublingual administration of this agent is rapid, allowing for fast action of the drug, and readily accomplished by a non-medically trained practitioner under medical supervision; by an MD or other medically licensed practitioner; and may be safely administered under close supervision in an at-home program. Intramuscular (IM) administration is a rapid acting method for ameliorating the symptoms as described herein. No other methods of administration are excluded from the Invention.

Cannabis is a medicinal plant that naturally produces bioactive compounds, including cannabinoids. Cannabis and cannabinoids are generally non-toxic and have minimal adverse side effects. This invention contemplates the use of all cannabis species and subspecies and the use of all cannabinoids produced by cannabis species and subspecies. A majority of U.S. states recognize the therapeutic value of cannabis and have legalized its medicinal use.

### Ketamine and Norketamine

The methods of the invention include the administration of ketamine, norketamine, 6-hydroxynorketamine, or a pharmaceutically acceptable salt thereof.

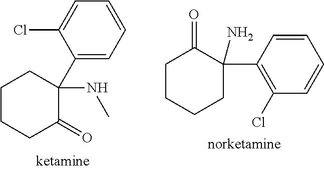 [SMILES: CNC1(c2ccccc2Cl)CCCCC1=O ; NC1(c2ccccc2Cl)CCCCC1=O]

As used herein, the term “ketamine” includes ketamine in its racemic (R/S) form, in its R-(−) enantiomerically pure form, or in its S-(+) enantiomerically pure form.

As used herein, the term “norketamine” includes norketamine in its racemic (R/S) form, in its R-(−) enantiomerically pure form, or in its S-(+) enantiomerically pure form.

As used herein, “enantiomerically pure” refers to compositions consisting substantially of a single isomer (i.e., substantially free of the opposite isomer), preferably consisting of 90%, 92%, 95%, 98%, 99%, or 100% (w/w) of a single isomer. For example, when the methods of the invention include the administration of enantiomerically pure R-(−)-ketamine, the pharmaceutical composition administered can include at least 95% (w/w) S-(+)-ketamine, and less than 5% (w/w) R-(−)-ketamine.

Ketamine racemate is primarily used for the induction and maintenance of general anesthesia. Enantiomerically pure S-(+)-ketamine (aka esketamine) is available for medical use, administered either IV (intravenously) or IM (intramuscularly), under the brand name KETANEST®. Enantiomerically pure R-(−)-ketamine is also known as arketamine. Ketamine is converted metabolically through demethylation to norketamine, in vivo, at rates dependent on the route of administration. For use in anesthesia, S-(+)-ketamine has been reported to be twice as potent as R-(−)-ketamine, and norketamine has been reported to have one third the potency of ketamine (C. S. T. Aun, Br. J. Anaesthesia 83: 29-41 (1999)).

As used herein, the term “6-hydroxynorketamine” includes 6-hydroxynorketamine in any of its 2R,6R; 2S,6S; 2S,6R; and 2R,6S isomerically pure forms (shown below).

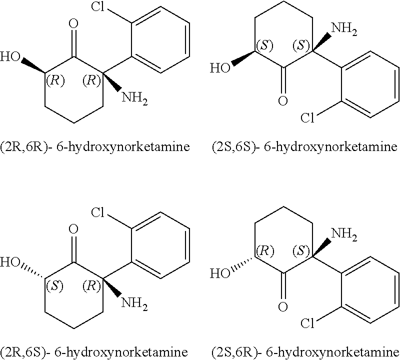

As used herein, “isomerically pure” refers to compositions consisting substantially of a single diastereomer (i.e., substantially free of other isomers), preferably consisting of 90%, 92%, 95%, 98%, 99%, or 100% (w/w) of a single isomer. For example, when the methods of the invention include the administration of isomerically pure (2R,6R)-6-hydroxynorketamine, the pharmaceutical composition administered can include at least 95% (w/w) (2R,6R)-6-hydroxynorketamine, and less than 5% (w/w) other isomers of 6-hydroxynorketamine. Alternatively, the methods of the invention include the administration of isomerically pure (2S,6S)-6-hydroxynorketamine, the pharmaceutical composition administered can include at least 95% (w/w) (2S,6S)-6-hydroxynorketamine, and less than 5% (w/w) other isomers of 6-hydroxynorketamine.

### Cannabis and Cannabinoid Compounds

The methods of the invention include the administration of cannabis. Cannabis, commonly known as marijuana, is a genus of flowering plants including at least three species: Cannabis sativa, Cannabis indica, and Cannabis ruderalis. Cannabis speciation is determined by plant phenotypes and secondary metabolite profiles. Modern advances in cannabis cultivation have led to the development of sub-species or “strains” of Cannabis with properties of both sativa and indica species. Such strains are referred to herein as “hybrid strains”.

Cannabinoids are a group of compounds produced by cannabis species that activate cannabinoid receptors (i.e., CB1 and CB2) in cells. Certain cannabinoids are psychoactive. At least 113 different cannabinoids have been identified in cannabis plants. Cannabinoids may easily cross the blood-brain barrier and have few side effects. The most notable cannabinoids are (−)-trans-49-tetrahydrocannabinol (delta-9-THC), (−)-trans-48-tetrahydrocannabinol (delta-8-THC), cannabinol (CBN), tetrahydrocannabivarin (THCV), and cannabidiol (CBD).

The methods of the invention include the administration of any cannabinoid containing product manufactured from any strain of cannabis, as well as administration of a synthetic cannabinoid.

### Formulation of Pharmaceutical Compositions

The administration of ketamine, norketamine, 6-hydroxynorketamine, or a pharmaceutically acceptable salt thereof, and cannabis or a cannabinoid may be by any suitable means that results in relief of an emotional disorder. The ketamine, norketamine, 6-hydroxynorketamine, or a pharmaceutically acceptable salt thereof, and cannabis or a cannabinoid may be contained in any appropriate amount in any suitable carrier substance, and is generally present in an amount of 1-95% by weight of the total weight of the composition. The composition may be provided in a dosage form that is suitable for the sublingual, buccal, oral, parenteral (e.g., intravenously, intramuscularly), pulmonary, intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, vapors, or aerosols. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Administration of the ketamine, norketamine, 6-hydroxynorketamine, or a pharmaceutically acceptable salt thereof, and cannabis or a cannabinoid in the form of a controlled release formulation is especially preferred in cases in which the active compound, either alone or in combination with a second agent, at therapeutic levels produces unwanted side effects, such as nausea.

Any of a number of strategies can be pursued in order to obtain controlled release of the active compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes.

### Sublingual and Buccal Dosage Forms

Formulations for sublingual may be in the form of films, strips, lozenges, and orally dissolving tablets. An orally dissolving tablet (ODT) refers to pharmaceutical dosage form designed to be dissolved on the tongue rather than swallowed whole, or designed to dissolve on the sublingual or buccal mucosa for sublingual or mucosal administration. Alternatively, the dosage form can be a lozenge (for slower administration as the lozenge dissolved over the course of 5-10 minutes), or as a rapidly dissolving film (dissolving over the course of less than 2 minutes). The active compound is administered via absorption in the mouth (i.e., buccally or sublingually). The formulation excipients are edible and pharmaceutically acceptable using excipients known in the art for the preparation of films, strips, lozenges, and orally dissolving tablets. For example, a film is prepared typically using hydrophilic polymers that rapidly dissolves on the tongue, palatine tissue, or buccal cavity, delivering the active compound to the systemic circulation via dissolution when contact with liquid is made.

### Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active compound in a predetermined pattern (e.g., in order to achieve a controlled release formulation) or it may be adapted not to release the active compound until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active compound). The coating may be applied on the solid dosage form in a similar manner as that described in Encyclopedia of Pharmaceutical Technology, supra.

For combination therapies, two drugs may be mixed together in the tablet, or may be partitioned. In one example, the first drug is contained on the inside of the tablet, and the second drug is on the outside, such that a substantial portion of the second drug is released prior to the release of the first drug.

Formulations for oral use may also be presented as chewable tablets, or as hard gelatin capsules wherein the active compound is mixed with an inert solid diluent (e.g., potato starch, lactose, microcrystalline cellulose, calcium carbonate, calcium phosphate or kaolin), or as soft gelatin capsules wherein the active compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment. Formulations for oral use can include cannabis or a cannabinoid in the form of a food (e.g., a gummy bear or cookie infused with cannabis), or in the form of a beverage.

### Pulmonary Administration

Cannabis is commonly administered via pulmonary administration, e.g., by smoking or vaporizing cannabis plant material, cannabis oil, or cannabis concentrates. For example, cannabis plant material and may be rolled into a cigarette and smoked, or smoked from a pipe or other implement. Cannabis plant material, oil, or concentrate may be placed in an electronic inhalation device, sometimes generically referred to as a vaporizer or electronic cigarette, which vaporizes and/or aerosolizes cannabis compounds, e.g., cannabinoids, in order to deliver said compounds in an inhalable form.

### Controlled Release Oral Dosage Forms

Controlled release compositions for oral use may, e.g., be constructed to release the active compound by controlling the dissolution and/or the diffusion of the active drug substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Liquids for Oral Administration

Powders, dispersible powders, or granules suitable for preparation of an aqueous suspension by addition of water are convenient dosage forms for oral administration. Formulation as a suspension provides the active compound in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

### Parenteral Compositions

The pharmaceutical composition containing ketamine, norketamine, 6-hydroxynorketamine, or a pharmaceutically acceptable salt thereof may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active compound, the composition may include suitable parenterally acceptable carriers and/or excipients. The active drug(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the suitable active compound is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate).

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices.

Materials for use in the preparation of microspheres and/or microcapsules are, e.g., biodegradable/bioerodible polymers such as polyglactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

### Rectal Compositions

For rectal application, suitable dosage forms for a composition include suppositories (emulsion or suspension type), and rectal gelatin capsules (solutions or suspensions). In a typical suppository formulation, the active compound is combined with an appropriate pharmaceutically acceptable suppository base such as cocoa butter, esterified fatty acids, glycerinated gelatin, and various water-soluble or dispersible bases like polyethylene glycols and polyoxyethylene sorbitan fatty acid esters. Various additives, enhancers, or surfactants may be incorporated.

### Vaginal Compositions

For vaginal application, suitable dosage forms for a composition include suppositories (emulsion or suspension type), and vaginal gelatin capsules (solutions or suspensions). In a typical suppository formulation, the active compound is combined with an appropriate pharmaceutically acceptable suppository base such as cocoa butter, esterified fatty acids, glycerinated gelatin, and various water-soluble or dispersible bases like polyethylene glycols and polyoxyethylene sorbitan fatty acid esters. Various additives, enhancers, or surfactants may be incorporated.

### Intranasal and Inhalation Compositions

For administration by inhalation, typical dosage forms include nasal sprays and aerosols. In a typically nasal formulation, the active compound is dissolved or dispersed in a suitable vehicle. The pharmaceutically acceptable vehicles and excipients (as well as other pharmaceutically acceptable materials present in the composition such as diluents, enhancers, flavoring agents, and preservatives) are selected in accordance with conventional pharmaceutical practice in a manner understood by the persons skilled in the art of formulating pharmaceuticals.

### Percutaneous and Topical Compositions

The pharmaceutical compositions may also be concurrently administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

Examples of emulsifying agents are naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE™. Examples of chelating agents are sodium EDTA, citric acid, and phosphoric acid. Examples of gel forming agents are CARBOPOL™, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN™)).

The compositions may be adapted for direct application or for introduction into relevant orifice(s) of the body (e.g., rectal, urethral, vaginal or oral orifices). The composition may be applied by means of special drug delivery devices such as dressings or alternatively plasters, pads, sponges, strips, or other forms of suitable flexible material.

### Dosages

The dosage of ketamine, norketamine, 6-hydroxynorketamine, or a pharmaceutically acceptable salt thereof, and cannabis or a cannabinoid to be concurrently administered depends on several factors, including: the administration method, the condition or symptom to be treated, the severity of the condition or symptom, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

As described above, the active compounds may be concurrently administered orally in the form of tablets, capsules, elixirs or syrups; or vaginally or rectally in the form of suppositories. Parenteral administration of the active compound is suitably performed, for example, in the form of saline solutions or with the compound incorporated into liposomes. Sublingual or buccal administration of the active compound may be in the form of films, strips, lozenges, and orally dissolving tablets.

The amount of ketamine, norketamine, 6-hydroxynorketamine, or a pharmaceutically acceptable salt thereof, administered can be from about 0.01 mg of active compound per kg of the subject's body weight (mg/kg) to about 5 mg/kg (e.g., from about 0.05 mg/kg to about 1 mg/kg, from about 0.05 mg/kg to about 0.5 mg/kg, from about 1 mg/kg to about 3 mg/kg, or from about 2 mg/kg to about 5 mg/kg), depending upon the route of administration. In general, the dose will be in the range of about 10 mg/day to about 300 mg/day (e.g., from about 25 mg/day to about 150 mg/day or from about 50 mg/day to about 300 mg/day) for sublingual administration. The dose will be in the range of about 5 mg/day to about 100 mg/day (e.g., from about 5 mg/day to about 50 mg/day or from about 25 mg/day to about 100 mg/day) for intranasal administration. The dose will be in the range of about 2 mg/day to about 75 mg/day (e.g., from about 2 mg/day to about 35 mg/day or from about 25 mg/day to about 75 mg/day) for intravenous administration. The dose will be in the range of about 10 mg/day to about 75 mg/day (e.g., from about 10 mg/day to about 45 mg/day or from about 40 mg/day to about 75 mg/day) for intramuscular administration. The dose will be in the range of about 50 mg/day to about 250 mg/day (e.g., from about 50 mg/day to about 125 mg/day or from about 100 mg/day to about 250 mg/day) for transdermal administration.

The amount of cannabinoid administered can be in the range of 1 mg of cannabinoid to 1,000 mg of cannabinoid (e.g., from 1 mg to 50 mg, 2 mg to 9 mg, 5 mg to 15 mg, 10 mg to 30 mg, 15 mg to 50 mg, 25 mg to 250 mg, 50 mg to 500 mg, 250 mg to 750 mg, or 750 mg to 1.00 mg of cannabinoid), depending on the cannabinoid and route of administration. For example, if cannabis plant matter is administered, e.g., via pulmonary administration, the amount may be in the range of 0.05 g of cannabis plant matter to 1 g of plant matter per subject, e.g., 0.1 g of plant matter to 0.9 g of plant matter, 0.2 g of plant matter to 0.8 g of plant matter, 0.3 g of plant matter to 0.7 g of plant matter, 0.4 g of plant matter to 0.6 g of plant matter, or 0.5 g of plant matter. If cannabis extract or concentrate, or cannabis-containing foodstuff, is administered, the dose may be in the range of 1 mg of THC to 10 mg of THC (e.g., from 1 mg to 50 mg, 2 mg to 9 mg, 5 mg to 15 mg, 10 mg to 30 mg, 15 mg to 50 mg), depending on route of administration.

### Therapy

Therapy according to the invention may be provided at home, the doctor's office, a clinic, a hospital's outpatient department, or a hospital. Treatment begins at the physician's office so that the doctor can observe the therapy's effects closely and make any adjustments that are needed. The duration of the therapy depends on the type and severity of the emotional disorder being treated, the age and condition of the patient, the stage and type of the patient's emotional disorder, and how the patient responds to the treatment.

Optionally, the ketamine, norketamine, 6-hydroxynorketamine, or a pharmaceutically acceptable salt thereof, and cannabis or a cannabinoid is concurrently administered in combination with an additional agent (e.g., a muscle relaxant or an anti-inflammatory agent). For combination therapies, the dosage, frequency and mode of administration of each component of the combination can be controlled independently. For example, ketamine, norketamine, 6-hydroxynorketamine, or a pharmaceutically acceptable salt thereof, and cannabis or a cannabinoid may be concurrently administered sublingually in a regimen described herein, while the additional agent may be administered orally once per day. The combination of therapeutic agents may also be formulated together such that one administration delivers all actives.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Treatment of an Emotional Disorder Via Combination Therapy with Ketamine and cannabis

Use of a combination of ketamine and THC containing cannabis in the Center for Transformational Psychotherapy clinic has occurred in hundreds of ketamine assisted psychotherapy sessions over several years—both in office and at home—in combination with sublingual ketamine lozenges and preceding intra-muscular injections. This practice has been possible within the legal framework for cannabis in the State of California. Administration of cannabis has been through use of commercial vaporizers, with full consent of the involved patients.

One of our embodiments will include cannabis in a form that dissolves co-terminously with ketamine, with the duration of effect being similar, or longer for the cannabis effect than the ketamine effect. Cannabis also may be administered to subjects in small amounts via commercial vaporizers just prior to ketamine administration via sublingual lozenge, and nasal insufflation. For many patients who are anxious preceding a ketamine session, which is dissociative in nature and may be disorienting, cannabis provides a relaxation into the ketamine experience. Cannabis modification of the ketamine experience is reported as making the ketamine experience more colorful, and more open-hearted. Cannabis is reported as providing a smoother return to this reality. Cannabis is also reported to enhance the depth of the ketamine experience, particularly with respect to deep relaxation and the letting go of trauma. For some patients who have a limited ketamine experience, cannabis enhances ketamine's effects with respect to depression and obsessional states. Overall, patients report that the combination of cannabis and ketamine provides better outcomes for the treatment of anxiety, depression, and other emotional disorders, or provide treatment with reduced sedation relative to ketamine alone.

### Example 2: Combination Therapy with Ketamine and Delta-8-THC in a Sublingual Dosage

In some embodiments, ketamine is combined with delta-8-THC in a sublingual dosage form to treat an emotional disorder (e.g., reducing the severity of depression and/or anxiety). The effect of the combination therapy can include increasing feelings of comfort, conviviality, relaxation, and/or positive affect in the subject. The dosage form may be formulated, e.g., as a sublingual lozenge containing about 200 mg S-(+)-ketamine and about 40 mg of delta-8-THC. The dosage form can be administered to a subject once or twice daily to ameliorate symptoms of an emotional disorder.

### Example 3: Combination Therapy with Ketamine and Delta-8-THC in an Oral Dosage

In some embodiments, ketamine is combined with delta-8-THC in an oral dosage form to treat an emotional disorder (e.g., reducing the severity of depression and/or anxiety). The effect of the combination therapy can include increasing feelings of comfort, conviviality, relaxation, and/or positive affect in the subject. The dosage form may be formulated, e.g., as a capsule, containing about 200 mg S-(+)-ketamine and about 40 mg of delta-8-THC. The dosage form can be administered to a subject once or twice daily to ameliorate symptoms of an emotional disorder.

### Example 4: Combination Therapy with Ketamine and Delta-8-THCV in a Sublingual Dosage

In some embodiments, ketamine is combined with delta-8-THCV in a sublingual dosage form to treat an emotional disorder (e.g., reducing the severity of depression and/or anxiety). The effect of the combination therapy can include increasing feelings of comfort, conviviality, relaxation, and/or positive affect in the subject. The dosage form may be formulated, e.g., as a sublingual lozenge containing about 200 mg S-(+)-ketamine and about 25 mg of delta-8-THCV. The dosage form can be administered to a subject once or twice daily to ameliorate symptoms of an emotional disorder.

### Example 5: Combination Therapy with Ketamine and Delta-8-THCV in an Oral Dosage

In some embodiments, ketamine is combined with delta-8-THCV in an oral dosage form to treat an emotional disorder (e.g., reducing the severity of depression and/or anxiety). The effect of the combination therapy can include increasing feelings of comfort, conviviality, relaxation, and/or positive affect in the subject. The dosage form may be formulated, e.g., as a capsule, containing about 200 mg S-(+)-ketamine and about 25 mg of delta-8-THCV. The dosage form can be administered to a subject once or twice daily to ameliorate symptoms of an emotional disorder.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of treating an emotional disorder in a subject in need thereof, the method comprising administering to the subject (i) ketamine, norketamine, 6-hydroxynorketamine, or a pharmaceutically acceptable salt thereof, and (ii) cannabis or a cannabinoid, each in an amount that together is effective for the treatment of the emotional disorder.

2. The method of claim 1, wherein the emotional disorder is an anxiety disorder.

3. The method of claim 2, wherein the anxiety disorder is selected from Generalized Anxiety Disorder (GAD), Panic Disorder (PD), Social Anxiety Disorder (SAD), Obsessive-Compulsive Disorder (OCD), Specific Phobic Disorders (SPD), Post Traumatic Stress Disorder (PTSD), Agoraphobia, or Separation Anxiety Disorder (SeAD).

4. The method of claim 1, wherein the emotional disorder is a depressive disorder.

5. The method of claim 4, wherein the depressive disorder is selected from dysthymia, major depression, Bipolar II disorder, Postpartum depression, or adjustment disorder with depression.

6. The method of claim 1, wherein the method comprises ameliorating one or more physical symptoms associated with the emotional disorder, wherein the physical symptoms are selected from headache, migraine, physical symptoms related to the menstrual cycle such as breast tenderness, cramps and headaches, symptoms of somatization disorders, fibromyalgia, various neurologic symptoms and disorders, and pain from a wide variety of causes.

7. The method of any one of claims 1-6, wherein the cannabis is prepared from a strain of cannabis selected from a Cannabis indica species, a Cannabis sativa species, a hybrid species, a high-tetrahydrocannabinol (THC) species of cannabis, or a high-cannabidiol (CBD) species of cannabis.

8. The method of any one of claims 1-6, wherein administration of the cannabis or a cannabinoid to the subject is by a route selected from oral, pulmonary, or transdermal administration.

9. The method of any one of claims 1-6, wherein the administration of the ketamine, norketamine, 6-hydroxynorketamine, or a pharmaceutically acceptable salt thereof, and cannabis or a cannabinoid is by a route selected from oral, sublingual, intranasal, intramuscular, intravenous, transdermal, and rectal administration.

10. The method of any one of claims 1-6, wherein the ketamine, norketamine, 6-hydroxynorketamine, or pharmaceutically acceptable salt thereof, is formulated in a lozenge.

11. The method of any one of claims 1-6, wherein the ketamine, norketamine, 6-hydroxynorketamine, or a pharmaceutically acceptable salt thereof, and cannabis or a cannabinoid are concurrently administered once or more daily, once or more every other day, or once or more every three days, depending on the presence and/or severity of the emotional disorder or the physical symptoms of the emotional disorder.

12. The method of claims 1-6, wherein the method comprises initiating treatment following the appearance of the emotional disorder or the physical symptoms of the emotional disorder in the subject.

13. The method of claims 1-6, wherein the ketamine, norketamine, 6-hydroxynorketamine, or a pharmaceutically acceptable salt thereof, and cannabis or a cannabinoid are concurrently administered once or more daily, once or more every other day, or once or more every three days, depending on the presence and/or severity of the emotional disorder or the physical symptoms of the emotional disorder.

14. The method of any one of claims 1-6, wherein the concurrent administration is once daily for a period of from 1 to 10 days, followed by a period of at least two weeks during which no ketamine, norketamine, 6-hydroxynorketamine, or a pharmaceutically acceptable salt thereof, and cannabis or a cannabinoid are concurrently administered to the subject.

15. The method of claims 1-6, wherein the concurrent administration is once daily for a period of from 1 to 8 days, followed by a period of at least two weeks during which no ketamine, norketamine, 6-hydroxynorketamine, or a pharmaceutically acceptable salt thereof, and cannabis or a cannabinoid are concurrently administered to the subject.

16. The method of any one of claims 1-6, wherein the ketamine, norketamine, 6-hydroxynorketamine, or a pharmaceutically acceptable salt thereof, and cannabis or a cannabinoid are concurrently administered to the subject once or more daily and/or in the evening, or in a program of frequency related to the needs of the patient for relief from the emotional disorder or the physical symptoms of the emotional disorder.

17. The method of claims 1-6, wherein the method comprises administering an average daily dose of from 10 mg to 300 mg of racemic ketamine, or a pharmaceutically acceptable salt thereof, to the subject.

18. The method of claims 1-6, wherein the method comprises administering one or more doses of from 1 mg to 300 mg of norketamine, or a pharmaceutically acceptable salt thereof, to the subject.

19. The method of claims 1-6, wherein the method comprises administering an average daily dose of from 10 mg to 300 mg of enantiomerically pure S-(+)-norketamine, or a pharmaceutically acceptable salt thereof, to the subject.

20. The method of claims 1-6, wherein the method comprises administering an average daily dose of from 10 mg to 300 mg of enantiomerically pure R-(−)-norketamine, or a pharmaceutically acceptable salt thereof, to the subject.

21. The method of claims 1-6, wherein the method comprises administering an average daily dose of from 10 mg to 300 mg of racemic norketamine, or a pharmaceutically acceptable salt thereof, to the subject.

22. The method of claims 1-6, wherein the method comprises administering one or more doses of from 1 mg to 300 mg of 6-hydroxynorketamine, or a pharmaceutically acceptable salt thereof, to the subject.

23. The method of claims 1-6, wherein the method comprises administering an average daily dose of from 10 mg to 300 mg of isomerically pure (2R,6R)-6-hydroxynorketamine, or a pharmaceutically acceptable salt thereof, to the subject.

24. The method of claims 1-6, wherein the method comprises administering an average daily dose of from 10 mg to 300 mg of isomerically pure (2S,6S)-6-hydroxynorketamine, or a pharmaceutically acceptable salt thereof, to the subject.

25. The method of any one of claims 1-24, wherein (i) the ketamine, norketamine, 6-hydroxynorketamine, or a pharmaceutically acceptable salt thereof, and (ii) the cannabis or a cannabinoid are administered separately within 30 minutes of each other.

26. The method of any one of claims 1-24, wherein (i) the ketamine, norketamine, 6-hydroxynorketamine, or a pharmaceutically acceptable salt thereof, and (ii) the cannabis or a cannabinoid are administered concurrently.

27. The method of claim 26, wherein (i) the ketamine, norketamine, 6-hydroxynorketamine, or a pharmaceutically acceptable salt thereof, and (ii) the cannabis or a cannabinoid are formulated together and administered simultaneously.

28. The method of claim 27, wherein (i) the ketamine, norketamine, 6-hydroxynorketamine, or a pharmaceutically acceptable salt thereof, and the cannabis or a cannabinoid are formulated together in a sublingual dosage form; and (ii) the sublingual dosage form is administered sublingually.

29. The method of claim 27, wherein the sublingual dosage form is selected from films, strips, lozenges, and orally dissolving tablets.

30. The method of any one of claims 1-29, wherein the cannabis or cannabinoid comprises at least one of delta-9-THC, delta-8-THC, cannabinol (CBN), tetrahydrocannabivarin (THCV), cannabidiol (CBD), and cannabivarin (CBV).

31. The method of claim 30, wherein the method includes administering one or more doses of from 1 mg to 40 mg of delta-9-THC to the subject.

32. The method of claim 30, wherein the method includes administering an average daily dose of from 2 mg to 100 mg delta-9-THC to the subject.

33. The method of claim 30, wherein the method includes administering one or more doses of from 1 mg to 80 mg of delta-8-THC to the subject.

34. The method of claim 30, wherein the method includes administering an average daily dose of from 2 mg to 100 mg delta-8-THC to the subject.

35. The method of claim 30, wherein the method includes administering one or more doses of from 1 mg to 30 mg of CBN to the subject.

36. The method of claim 30, wherein the method includes administering an average daily dose of from 2 mg to 100 mg CBN to the subject.

37. The method of claim 30, wherein the method includes administering one or more doses of from 1 mg to 30 mg of delta-9-THCV to the subject.

38. The method of claim 30, wherein the method includes administering an average daily dose of from 2 mg to 100 mg delta-9-THCV to the subject.

39. The method of claim 30, wherein the method includes administering one or more doses of from 1 mg to 30 mg of delta-8-THCV to the subject.

40. The method of claim 30, wherein the method includes administering an average daily dose of from 2 mg to 100 mg delta-8-THCV to the subject.

41. The method of claim 30, wherein the method includes administering one or more doses of from 10 mg to 800 mg of CBD to the subject.

42. The method of claim 30, wherein the method includes administering an average daily dose of from 20 mg to 1,000 mg CBD to the subject.

43. The method of claim 30, wherein the method includes administering one or more doses of from 1 mg to 30 mg of CBV to the subject.

44. The method of claim 30, wherein the method includes administering an average daily dose of from 2 mg to 100 mg CBV to the subject.
